(19) United States
(12) Patent Application Publication
(10) Pub. No.: US 2014/0194732 A1
(43) Pub. Date: Jul. 10, 2014

(54) TROCAR, AND SURGERY ASSISTANCE SYSTEM

(71) Applicants: National University Corporation Chiba University, Chiba-shi (JP); Advanced Healthcare Co., Ltd., Tokyo (JP)

(72) Inventor: Toshiya NAKAGUCHI, Funabashi-shi (JP)

(73) Assignees: National University Corporation Chiba University, Chiba-shi (JP); Advanced Healthcare Co., Ltd., Tokyo (JP)

(21) Appl. No.: 13/974,780

(22) Filed: Aug. 23, 2013

(30) Foreign Application Priority Data
Jan. 10, 2013 (JP) 2013-002338

(51) Int. Cl. A61B 17/34; A61B 5/06; A61B 17/29

(52) U.S. Cl. CPC A61B 17/3415; A61B 17/29; A61B 5/064
USPC 600/424

(57) ABSTRACT

Problem

To provide a surgery assistance system that performs measurement of shapes within the abdominal cavity at high accuracy in three dimensions, and a trocar for use in such a surgery assistance system.

Means for Solution

A surgery assistance system 101 includes forceps trocars 1a and 1b having retractable cameras 17a and 17b and marks 19a and 19b, a laparoscope trocar 3, forceps 4a and 4b, a laparoscope 5 having a mark 19d, an image processing device 6 that inputs the images obtained from the retractable cameras 17a and 17b and the image obtained from the laparoscope 5 and combines these images to create a three dimensional image, a three dimensional monitor 7 that outputs the three dimensional image created by the image processing device 6, and an optical tracking sensor 9. The positional relationships of the marks 19 and the corresponding cameras 17 are fixed. The positions of the marks 19 are detected by the optical tracking sensor 9, and the image processing device 6 estimates the distance between the cameras.

### TECHNICAL FIELD

The present invention relates to a trocar and to a surgery assistance system that includes a trocar, and in particular relates to measurement of shapes in three dimensions.

### BACKGROUND ART

In recent years, in order to maintain and enhance the QOL (quality of life) of patients, surgical operations of low invasiveness are being performed using laparoscopic surgery and so on. In abdominal laparoscopic surgery, carbonic acid gas is injected into the abdominal cavity so that the abdominal wall is distended, and thereby space and a good field of view for manipulation are ensured. A small hole is formed in the abdominal wall, and an instrument called a trocar is inserted. Then, usually, a laparoscope (i.e. a CCD camera) and a forceps (which is a surgical instrument) are inserted into the interior of the body of the patient, and the required surgical operation is performed while observing an image displayed upon a monitor by the laparoscope.

Now this operation is typically performed while relying only upon the image obtained from the laparoscope. Ascertaining the position of the forceps within the abdominal cavity demands high experience from the surgeon. In particular, it is impossible to derive information related to depth from the image displayed upon the monitor, and so accordingly reliance upon the experience and the intuition of the surgeon for depth estimation is unavoidable. And there is a fear that erroneous contact with internal organs or the like may occur, if an inexperienced surgeon inserts the forceps too far.

In order to cope with this type of problem, a proximity-aware operation navigation system has been proposed (refer to Non Patent Document #1) which emits a warning when the forceps has been inserted too far inward on the basis of a position sensor that is provided to the forceps. Due to this, even if image information relating to depth is not derived, it is still possible to prevent erroneous contact due to the forceps being inserted too far.

### CITATION LIST

### Non-Patent Literature

Non-Patent Document #1: Yume Honda, Ikuma Sato, and Ryoichi Nakamura, “Evaluation of the Effectiveness of Surgical Navigation with Distance Sensation Indicator for Laparoscopic Surgery”, 20th Japan Computer Surgery Conference, Yokohama, 22 24 November 2011, Journal of Japan Society of Computer Aided Surgery, 13(3): 280 281, 2011.

### SUMMARY OF THE INVENTION

### Technical Problem

While the navigation system described above is capable of alleviating the burden upon the surgeon, it is not related to measurement of shapes in three dimensions.

In order to improve the field of view during laparoscopic surgery in an innovative manner, it is necessary to estimate depths within the abdominal cavity, to measure shapes in three dimensions, and to recreate this information upon a monitor display or the like.

Now, three dimensional endoscopes are currently manufactured and marketed, and it is possible to measure shapes within the abdominal cavity in three dimensions by using such a three dimensional endoscope. A three dimensional endoscope incorporates two cameras, and depth is estimated on the basis of the triangle defined by the two cameras and the subject point. However, with a three dimensional endoscope, the distance between the cameras is extremely small, and as a result the accuracy of depth estimation is not good.

The present invention has been conceived in order to solve the problem described above, and its object is to provide a surgery assistance system with which measurement of shapes in three dimensions within the abdominal cavity of a patient can be performed at high accuracy, and to provide a trocar that is used in such a surgery assistance system.

### Means for Solution

In order to solve the problem described above, the present invention proposes a trocar for being passed through an abdominal wall of a patient, comprising: a pipe portion that inserts a surgical instrument into the interior of the body of the patient; a head portion that is connected to an upper portion of said pipe portion; an opening portion that is provided at a position of said pipe portion that, during surgery, is within the body of the patient; a camera that is disposed so that it can be changed between a stored position in which it is stored within the pipe portion and a deployed position in which it is deployed via said opening portion to the exterior of the pipe portion and is capale of photography; and a position mark provided upon said head portion.

More desirably, said position mark is an optical mark.

According to the basic theory of three dimensional shape measurement, the depth estimation accuracy is enhanced if the accuracy of estimation of the distance between the cameras is improved. A retractable camera and a position mark are provided to the trocar of the present invention, and, even if the trocar wobbles, the positional relationship between the camera and the mark does not change. Accordingly, if the position mark is detected at high accuracy, then the position of the retractable camera can be estimated at high accuracy, and as a result it is also possible to estimate the depth at high accuracy.

And, in order to solve the problem described above, the present invention proposes a surgery assistance system including: a laparoscope including a camera and a position mark; a forceps trocar having a retractable camera that can be changed between a stored position and a deployed position, and a position mark; a position detection sensor that detects the positions of the position mark of said laparoscope and of the position mark of said forceps trocar; and an image processing device that estimates the positions of said cameras on the basis of the positions of said position marks, and that combines the images obtained from said cameras on the basis of the positions of said cameras to create a three dimensional image.

Generally, a plurality of forceps are used in laparoscopic surgery. As a result, apart from the laparoscope, a plurality of cameras are inserted into the abdominal cavity of the patient. Due to this, it is possible to estimate depths at high accuracy, and it is possible to perform measurement of shapes within the abdominal cavity in three dimensions at high accuracy.

Generally, with laparoscopic surgery, a plurality of trocars for the use of forceps are implanted in the abdominal wall as being spaced apart almost equally. To put it in another manner, the possibility that the trocars will be grouped closely together is almost nil. Due to this, it is possible to ensure sufficiently great distances between the cameras, and it is possible to estimate the depths at high accuracy, so that it is possible to perform measurement of the shapes within the abdominal cavity in three dimensions at high accuracy.

Moreover, in order to solve the problem described above, the present invention proposes a surgery assistance system including: a plurality of forceps trocars, each having a retractable camera that can be changed between a stored position and a deployed position, and a position mark; a position detection sensor that detects the positions of the position marks of said forceps trocars; and an image processing device that estimates the positions of said cameras on the basis of the positions of said position marks, and combines the images obtained from said cameras on the basis of the positions of said cameras to create a three dimensional image.

Due to this, it is possible to perform measurement of shapes within the abdominal cavity in three dimensions with good accuracy. Moreover the invasiveness is reduced, since no hole for laparoscope is required.

More desirably, this surgery assistance system further includes a three dimensional projector that is provided above the operating table, and that projects said three dimensional image upon the abdomen of the patient.

With this arrangement, the line of sight of the surgeon and the direction of his field of operation coincide with one another, so that he can really experience the same feeling as that during an open-abdomen operation.

In order to solve the problem described above, the present invention proposes a port for being passed through a chest wall of a patient, comprising: a pipe portion that inserts a surgical instrument into the interior of the chest of the patient; a head portion that is connected to an upper portion of said pipe portion; an opening portion that is provided at a position of said pipe portion that, during surgery, is within the body of the patient; a camera that is disposed so that it can be changed between a stored position in which it is stored within the pipe portion and a deployed position in which it is deployed via said opening portion to the exterior of the pipe portion and is capale of photography; and a position mark provided upon said head portion.

### Advantageous Effect of the Invention

According to the present invention, it is possible to perform measurement of shapes within the abdominal cavity of a patient at high accuracy in three dimensions.

### BRIEF DESCRIPTION OF THE DRAWINGS

FIG. 1 shows a first embodiment of the surgery assistance system;

FIG. 2A shows a trocar having a retractable camera and a mark;

FIG. 2B shows a trocar having a retractable camera and a mark (another point of view);

FIG. 3A shows a variant embodiment of the trocar (deployed position);

FIG. 3B shows a variant embodiment of the trocar (stored position);

FIG. 4 shows the fundamental theory of three dimensional shape measurement;

FIG. 5 illustrates the difficulty of camera position estimation;

FIG. 6A shows a relationship between camera distance (extremely small) and depth estimation accuracy;

FIG. 6B shows a relationship between camera distance (quite large) and depth estimation accuracy;

FIG. 7 is a second embodiment of the surgery assistance system; and

FIG. 8 is a third embodiment of the surgery assistance system.

### DESCRIPTION OF THE EMBODIMENTS

### The First Embodiment

(Structure of the Surgery Assistance System)

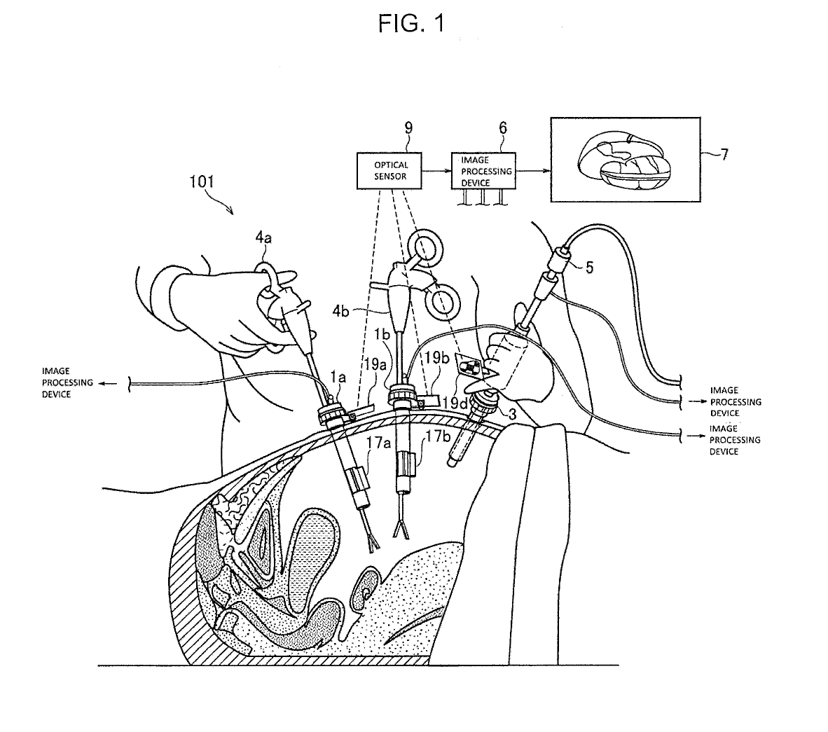

A surgery assistance system 101 that employs a three dimensional image will now be explained. FIG. 1 shows the general structure of this surgery assistance system 101.

The surgery assistance system 101 comprises forceps trocars 1a and 1b (to be described in detail hereinafter) that are respectively equipped with retractable cameras 17a and 17b and respectively carry marks 19a and 19b, a laparoscope trocar 3, forceps 4a and 4b, a laparoscope 5 that carries a mark 19d, an image processing device 6 that inputs images obtained from the retractable cameras 17a and 17b and an image obtained from the laparoscope 5 and performs processing to create a three dimensional image by combining these images, a three dimensional monitor 7 that outputs this three dimensional combined image created by the image processing device 6, and an optical tracking sensor 9.

The forceps 4a and 4b are surgical instruments of one type, and are used for grasping, holding down, pulling, and cutting blood vessels and organs and so on. Each of them is generally formed as a pair of scissors, and its inner working end portion is operated by outer gripping portions being rotated around a fulcrum. When the gripping portions are closed together, the forceps 4a, 4b can be inserted through the trocar 1a, 1b. It should be understood that while, generally, a plurality of forceps are used in abdominal laparoscopic surgery, at least one forceps and one forceps trocar are enough for application of this system.

The laparoscope 5 is an endoscopic instrument of one type, and comprises a camera and a light source. The laparoscope 5 is inserted into the body of the patient by being passed through the laparoscope trocar 3. The mark 19d is provided at a position on the laparoscope which is exterior to the body of the patient.

The optical tracking sensor 9 measures the positions of the marks 19a, 19b, and 19d in three dimensions, and outputs the results of these measurements to the image processing device 6. It should be understood that while, in this embodiment, the optical tracking sensor 9 is a device that recognizes black and white patterns on the marks with visible light radiation, it would also be acceptable to arrange for it to emit infrared radiation, and to receive infrared radiation reflected by the marks. Moreover, the use of an optical tracking sensor is not to be considered as being limitative; it would also be acceptable to employ a magnetic sensor, provided that it can measure the positions of the marks in three dimensions.

(Structure of the Trocars)

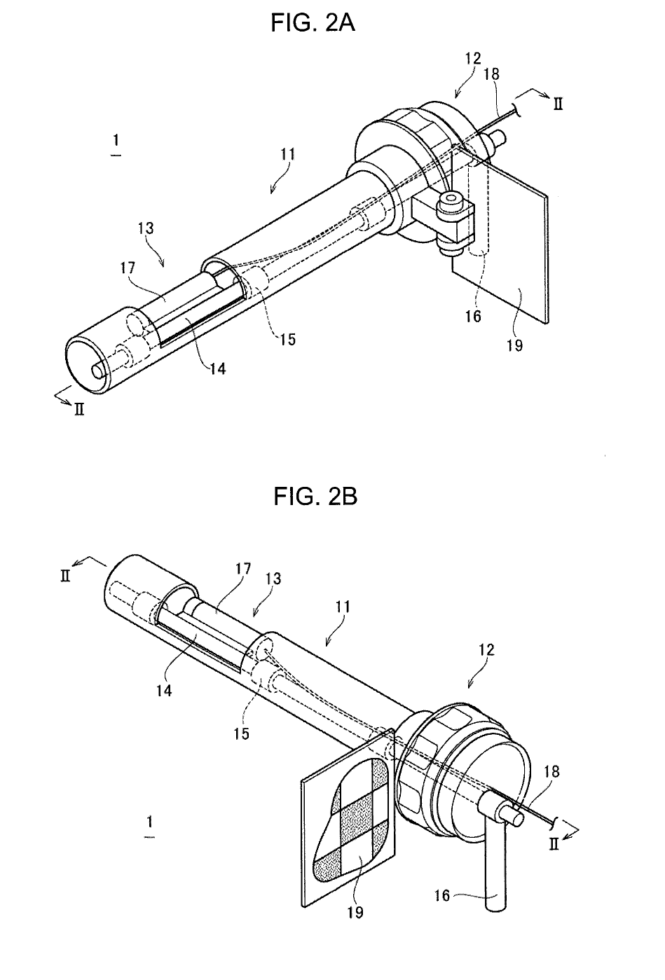

The structure of these trocars equipped with retractable cameras will now be described. FIG. 2 shows two perspective views of one of these trocars equipped with a retractable camera. FIG. 2A and FIG. 2B show this trocar from different points of view.

The trocar 1 comprises a pipe portion 11 and a head portion 12. The greater part of the pipe portion 11 is inserted into a hole in the abdominal wall of the patient. The head portion 12 is provided as connected to the upper portion of the pipe portion 12. The head portion 12 is hollow, and a forceps can be inserted thereinto from above. Moreover, although the details thereof are not shown, the head portion 12 is provided with a sealing mechanism that prevents air leakage when the forceps is inserted and withdrawn, and with an air blowing mechanism that injects air into the abdominal cavity.

An opening portion 13 is provided at a position of the pipe portion 11. The position of the opening portion 13 can be relied upon to be within the body of the patient when the trocar has been inserted. A shaft 14 is disposed along the axial direction of the pipe portion, along an edge of the opening portion 13. A plurality of bearings 15 are fixed to the inner wall of the pipe portion 11, and these bearings 15 hold the shaft 14 so that it can be rotated. The end portion of the shaft 14 projects to the exterior of the trocar. A selection lever 16 is fixed to the end portion of the shaft 14. This selection lever 16 can be rotated between a stored position and a deployed position, and can be retained in each of these positions. A detent mechanism not shown in the figures may be used to retain.

A camera 17 is rigidly and integrally fixed to the shaft 14 at a position that corresponds to the opening portion 13. A cable 18 is connected to the camera 17, and this cable 18 is led out through the trocar 1 and is connected to the external image processing device 6.

When the pipe portion 11 is to be inserted into a hole in the abdominal wall of the patient, the selection lever 16 is fixed in the stored position, and this holds the camera 17 in the stored position via the shaft 14. Due to this, it is possible to insert the pipe portion 11 through the hole in the abdominal wall without the camera 17 causing any hindrance. After the pipe portion 11 has been inserted, the selection lever 16 is moved over to the deployed position and fixed there, so that the camera 17 is moved to the deployed position via the shaft 14. Photography is performed in this state, and then, when the pipe portion 11 is to be withdrawn after the operation has been completed, the selection lever 16 is moved back to the stored position and is fixed there, so that the camera 17 is moved back to the stored position via the shaft 14. Due to this, it is possible to withdraw the pipe portion 11 from the hole in the abdominal wall without any hindrance being caused by the camera 17.

It should be understood that while, in the shown configuration, the cable 18 is led out along the shaft 18, it would be even more desirable to make the shaft 14 hollow, and to lead out the cable 18 through the shaft 14; in this case, there would be no danger of the cable 18 becoming disconnected during insertion of the forceps.

The mark 19 is provided upon the head portion 12 of the trocar. In this embodiment, a white and black checkered flag pattern is shown as one example of this mark 19, but this is not to be considered as being limitative; any mark that can be recognized by the optical tracking sensor 9 will be acceptable.

(A variant Embodiment of the Trocar)

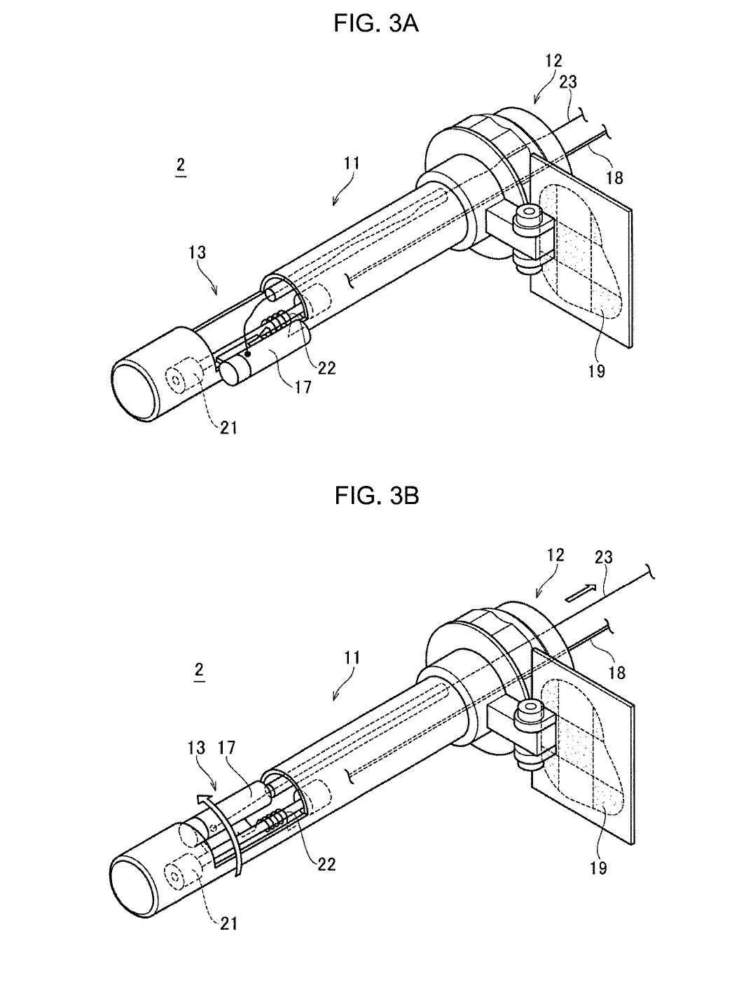

The structure described above is not limitative; it is only necessary that the trocar should be provided with a camera 17 and a mark 19. FIG. 3 is a perspective view of a trocar 2 according to a variant embodiment. FIG. 3A is a figure showing this trocar 2 in the deployed state with the camera 17 in the deployed position, while FIG. 3B is a figure showing this trocar 2 in the stored state with the camera 17 in the stored position. Elements of the structure that are the same as in the previously described embodiment are denoted by the same reference symbols. This trocar 2 comprises a pipe portion 11 and a head portion 12. An opening portion 13 is provided at a position of the pipe portion 11. The position of the opening portion 13 can be relied upon to be within the body of the patient when the trocar has been inserted. A rotatable hinge mechanism 21 is provided along an edge of the opening portion 13 and extends along the axial direction of the pipe portion 11, and a camera 17 is connected to the pipe portion 11 via this hinge mechanism 21. A torsion spring 22 is provided to the hinge mechanism 21, and normally the elastic force of this torsion spring 22 acts to deploy the camera 17. On the other hand, a tension cable 23 is connected to the camera 17 and extends to the exterior of the trocar, and, when this tension cable 23 is pulled, the camera 17 is stored in the opening portion 13 against the resistance of the elastic force of the torsion spring 22 which is overcome. And a cable 18 is connected to the camera 17.

When the pipe portion 11 is to be inserted into a hole in the abdominal wall of the patient, the tension cable 23 is pulled and the camera 17 is held in the stored position, and, after the pipe portion 11 has been inserted, the tension in the tension cable 23 is slackened, and the camera 17 moves over to the deployed position. Photography is performed in this state, and then, when the pipe portion 11 is to be withdrawn after the operation has been completed, the tension cable 23 is again pulled, so that the camera 17 is moved back to the stored position.

It should be understood that the cable 23 is protected by a guide, so as to reduce the danger of the tension cable 23 being cut when the forceps 4 is inserted into or pulled out from the trocar.

The mark 19 is provided upon the head portion. It should be understood that, for convenience, the checkerboard pattern is shown on the rear surface of the mark by dotted lines in the drawing.

(Measurement of Shapes in Three Dimensions)

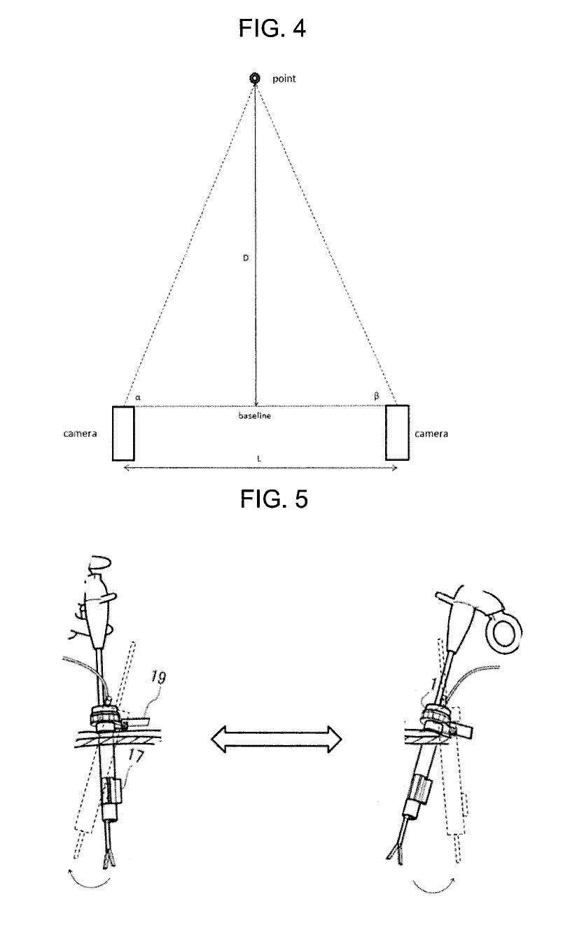

FIG. 4 is a conceptual figure showing the fundamental theory of measurement of shapes in three dimensions. The most important difference between measurement of shapes in two dimensions and in three dimensions is estimation of depth.

In the triangle defined by two cameras and a subject point, the depth D can be estimated on the basis of the distance L between the two cameras, the angle a subtended by the base line between the cameras and the line of sight of one of the cameras, and the angle 8 subtended by the base line between the cameras and the line of sight of the other camera. It should be understood that it is possible to enhance the accuracy of estimation by increasing the number of cameras, since a greater number of triangles are thereby defined.

With the trocar 1 of this embodiment, the marks 19 are fixed. Moreover, during surgery, the cameras 17 are fixed in their deployment positions. In other words, the positional relationships between the marks 9 and the corresponding cameras 17 do not change. Due to this, the image processing device 6 is able to estimate the three dimensional positions of the cameras 17a and 17b on the basis of the three dimensional positions of the marks 19a and 19b respectively. In a similar manner, the image processing device 6 is able to estimate the three dimensional position of the camera of the laparoscope 5 on the basis of the three dimensional position of the mark 19d. In other words, it is capable of estimating the distances between the cameras.

Furthermore, for each subject point, the angles α and β are measured, and thus it is possible to estimate the positions in depth of the subject points on the basis of the fundamental theory described above. And it is possible to measure the three dimensional shapes within the abdominal cavity by shifting the subject point and repeating the estimation of its depth position.

(Beneficial Effects of this System)

(1) Since laparoscopic surgery using this surgery assistance system 101 is based upon conventional laparoscopic surgery, and there is no great change in the method of operation, accordingly a surgeon is able to take advantage of his current fund of knowledge and experience relating to conventional operational technique.

(2) Moreover, this surgery assistance system 101 has a simple structure that employs an improved trocar (refer to FIG. 2 or FIG. 3), so that it is possible still to apply a surgery assistance system of an already existing type with a few simple improvements.

(3) Now, in conventional abdominal laparoscopic surgery according to the prior art, the field of view is narrow, since the surgery is performed while only relying upon the image obtained from the laparoscope. In particular, no image information is obtained related to depth. And, if a new hole is opened in the abdominal wall in order to insert another camera for performing three dimensional shape measurement with good accuracy, then the advantage of low invasiveness is lost.

However, in this embodiment, it is possible to insert a plurality of cameras into the abdominal cavity of the patient by using the trocars 1a and 1b that are equipped with the retractable cameras 17a and 17b respectively. Since trocars for forceps are used at this time, there is no need to make any new holes in the abdominal wall. Due to this, it is possible to measure shapes in three dimensions while maintaining the advantage of low invasiveness.

Moreover, the image processing device 6 creates a three dimensional image, and this three dimensional image is outputted upon the three dimensional monitor 7. Thus, by viewing the three dimensional monitor 7, the surgeon is able to obtain a broad field of view that includes depth information. Due to this, it is possible to alleviate the burden upon the surgeon.

It should be understood that, since it is possible to perform three dimensional measurement and to generate a three dimensional image, accordingly, naturally, it is also possible to perform two dimensional measurement and to generate a two dimensional image. And it is also possible to change over between a two dimensional image and a three dimensional image.

(Beneficial Effects Related to Enhancement of Accuracy)

(1) As explained in connection with the fundamental theory of three dimensional shape measurement, it is necessary to derive the three dimensional positions of the cameras 17a and 17b for estimating the depth. However, since the cameras 17a and 17b are located within the abdominal cavity, it is not possible to measure their positions directly. Moreover, the angles of the trocars 1a and 1b change somewhat along with the movement of the forceps 4a and 4b, so that, as a result, the cameras 17a and 17b quiver to a certain extent. Due to this, there is the problem that it is difficult to estimate their three dimensional positions. FIG. 5 is a conceptual figure for explanation of problems related to the difficulty of camera position estimation.

Accordingly, the present inventors have directed their attention to the fact that the cameras 17a and 17b undergo a certain degree of quivering along with the shaking of the trocars 1a and 1b, and, because of this, the marks 19a and 19b are provided upon the head portions 12 of the trocars 1a and 1b. In other words, the positional relationships of the cameras 17a and 17b with the positions at which the marks 19a and 19b appear do not change. And furthermore, the positions of the marks 19a and 19b in three dimensions can be detected at high accuracy by the optical tracking sensor 9. Accordingly, it is possible to estimate the positions in three dimensions of the cameras 17a and 17b at high accuracy on the basis of the positions in three dimensions of the marks 19a and 19b.

It should be understood that the mark 19d is provided upon the laparoscope 5 in order for movement of the camera of the laparoscope 5 due to quivering of its trocar 3 not to present any problem. Due to the provision of this mark 19d, it is possible to estimate the position in three dimensions of the camera of the laparoscope 5 at high accuracy.

The result of the facts that it is possible to estimate the three dimensional positions of the cameras at high accuracy, and that it is possible to estimate the distance between the cameras at high accuracy, is that it is possible to estimate depths at high accuracy, and that it is possible to measure the three dimensional shapes within the abdominal cavity at high accuracy.

(2) As explained in connection with the fundamental theory of three dimensional shape measurement, the accuracy of depth estimation is enhanced by increasing the number of cameras. Generally a plurality of forceps are used in laparoscopic surgery (for example, two through five). As a result, apart from the laparoscope 5, the plurality of cameras 17 are inserted into the abdominal cavity. Due to this, it is possible to estimate depth at high accuracy, and it is possible to perform measurement of shapes within the abdominal cavity in three dimensions at high accuracy.

(3) Now, it is also possible to perform measurement of shapes in three dimensions within the abdominal cavity of the patient if a three dimensional endoscope is used. However, with a three dimensional endoscope, the distance between the cameras is extremely small, so that the triangle described above becomes extremely long and thin, and as a result the accuracy of depth estimation is deteriorated.

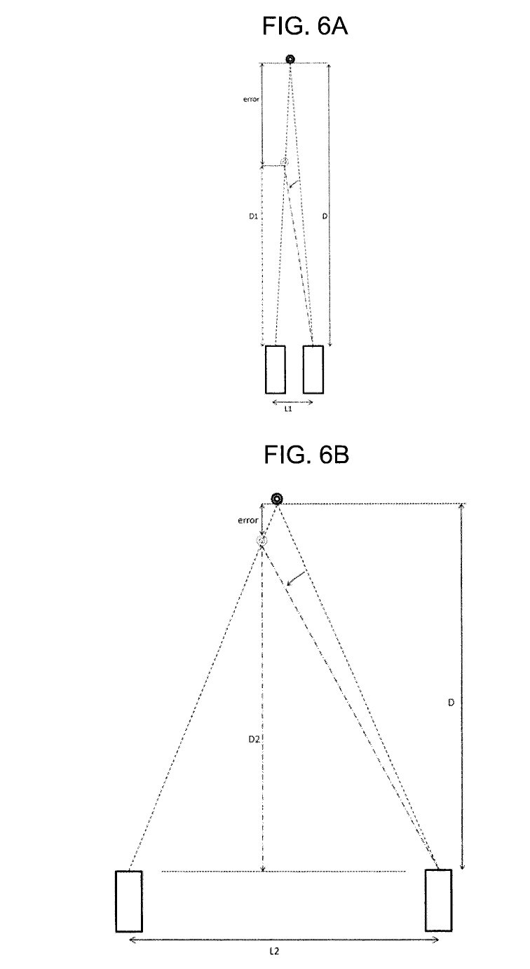

FIG. 6 is a conceptual figure showing the relationship between the distance between the cameras and the accuracy of depth estimation. FIG. 6A shows a case when the distance between the cameras is extremely small, while FIG. 6B shows a case when the distance between the cameras is quite large.

In FIG. 6A, the distance between the cameras is denoted by L1 and is extremely small, while the actual depth is denoted by D. If there is an error in the line of sight of one of the cameras, then the estimated depth becomes D1. And, in FIG. 6B, the distance between the cameras is denoted by L2 and is quite large, while the actual depth is denoted by D (the same as in FIG. 6A). If there is an error in the line of sight of one of the cameras (of the same level as in FIG. 6A), then the estimated depth becomes D2.

In this case, the error in the estimated depth D2 is small, by contrast with the estimated depth D1 that has a large error.

Generally, with laparoscopic surgery, a plurality of trocars 1 (for example two through five) for the use of forceps are implanted in the abdominal wall as being spaced apart almost equally. To put it in another manner, the possibility that the trocars 1 will be grouped closely together is almost nil. Due to this, it is possible to ensure sufficiently great distances between the cameras, and it is possible to estimate the depths at high accuracy, so that it is possible to perform measurement of the shapes within the abdominal cavity in three dimensions at high accuracy.

### The Second Embodiment

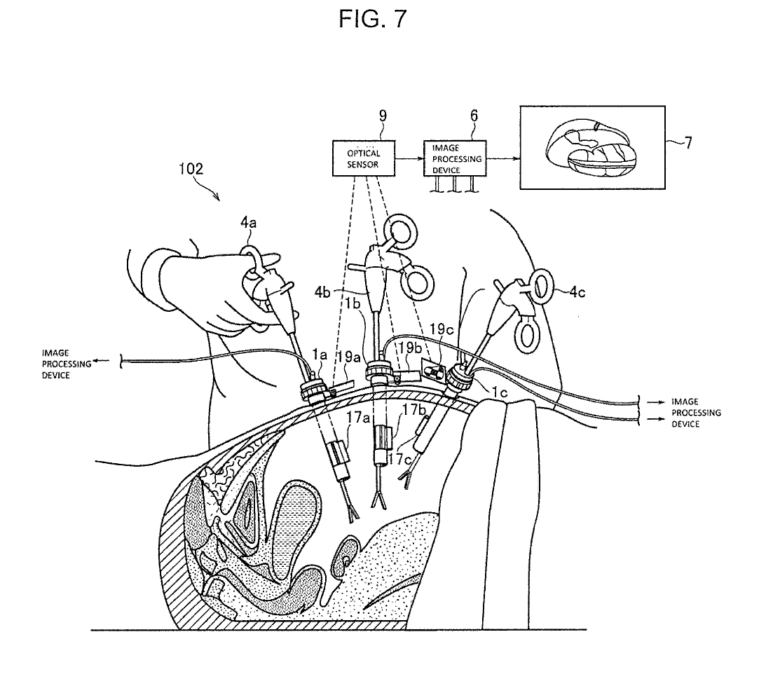

FIG. 7 is a figure showing the general structure of another surgery assistance system 102. This surgery assistance system 102 comprises forceps trocars 1a, 1b, and 1c that are respectively equipped with retractable cameras 17a, 17b, and 17c and marks 19a, 19b, and 19c, forceps 4a, 4b, and 4c, an image processing device 6 that estimates the three dimensional positions of the cameras 17a, 17b, and 17c on the basis of the three dimensional positions of the marks 19a, 19b, and 19c, combines the images obtained from the cameras, and creates a three dimensional image, and a three dimensional monitor 7 that outputs the three dimensional image created by the image processing device 6.

In other words, the laparoscope trocar 3, the laparoscope 5, and the mark 19d in the surgery assistance system 101 of the first embodiment are omitted, and another forceps trocar 1c including a retractable camera 17c, another forceps 4c, and a corresponding mark 19c are added instead.

It should be understood that while, generally, a plurality of forceps are used in abdominal laparoscopic surgery, at least two forceps and two forceps trocars are enough for the application of this system according to the second embodiment. Moreover it should be understood that, although no laparoscope is actually used in this embodiment, for convenience it is referred to as laparoscopic surgery.

By contrast to the situation when a laparoscope 5 is used as in the first embodiment, in which the surgeon needs actively to orient the laparoscope 5 in order to take a photograph of the spot where cutting or the like is being performed, since, in this embodiment, the retractable camera 17 reliably photographs the end portion of the forceps 4a, accordingly it is possible reliably to obtain an image of the actual spot where cutting or the like is being performed, which is a very important image. Thus, on the supposition that that the performance of the retractable camera 17 is high, it is possible to obtain an image of higher quality than that obtained with a laparoscope.

On the other hand, by dispensing with the laparoscope trocar 3 and the laparoscope 5, it becomes unnecessary to make any hole in the abdominal wall for passing laparoscope 5, so that the invasiveness becomes yet lower.

However, instead of the light source provided to the laparoscope 5, it is necessary to provide light sources to the trocars 1 (or to the cameras 17).

### The Third Embodiment

The third embodiment is a variant of the first and second embodiments. While in the first and second embodiments the surgeon performs an operation by manipulating the forceps 4 and the laparoscope 5 while looking at the monitor 7, there is a discrepancy between the line of sight of the surgeon and the direction towards the actual field of operation, so that the surgeon experiences a sense of discomfort, and this constitutes a burden. In particular, a surgeon who has performed a lot of open—abdomen operations sometimes finds it difficult to get used to abdominal laparoscopic surgery.

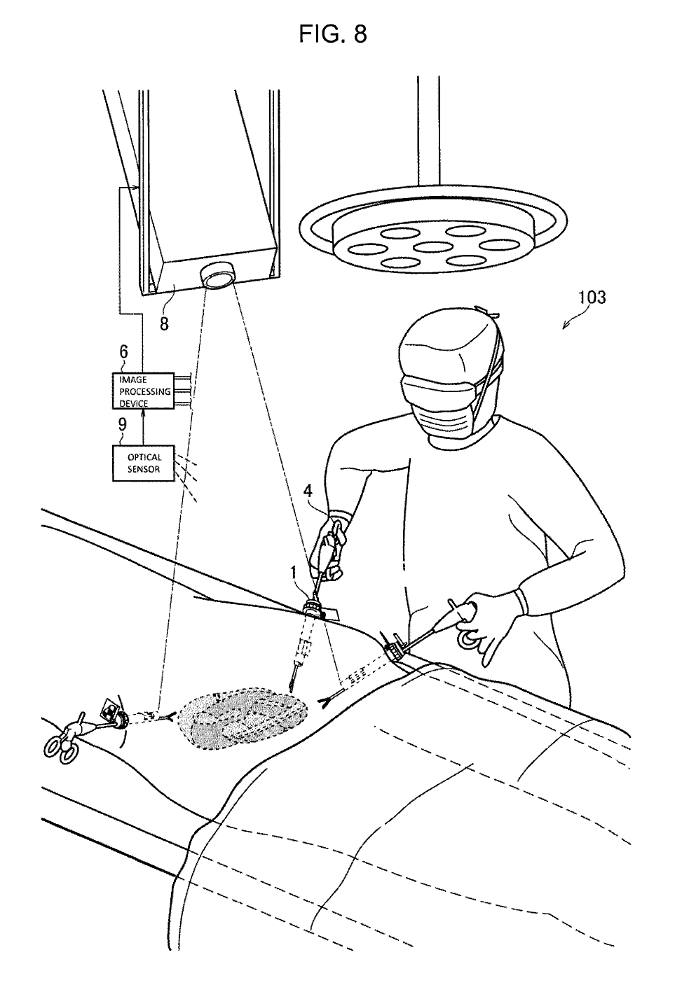

FIG. 8 is a figure showing the general structure of a surgery assistance system 103 according to this third embodiment. Elements that are common to the first and second embodiments are omitted as appropriate. This surgery assistance system 103 comprises a three dimensional projector 8, instead of the three dimensional monitor 7. The three dimensional projector 8 is provided over the operating table, and projects the combined image created by the image processing device 6 directly upon the abdominal portion of the patient.

Due to this, the line of sight of the surgeon and the direction of his field of operation coincide, so that he is able to experience the same feeling of reality as in an open-abdomen operation. This means that the burden upon the surgeon is alleviated.

<Port having Retractable Camera>

While the above explanation has been expressed in terms of abdominal laparoscopic surgery, the present invention can also be applied to chest laparoscopic surgery. However, the operating instrument that is termed a “trocar” in abdominal laparoscopic surgery is called a “port” in chest laparoscopic surgery. That is to say, a trocar and a port are devices of almost the same type.

### EXPLANATION OF THE REFERENCE SYMBOLS

1: trocar

2: trocar (variant embodiment)

3: trocar (for laparoscope)

4: forceps

5: laparoscope

6: image processing device

7: three dimensional monitor

8: three dimensional projector

9: optical tracking sensor

11: pipe portion

12: head portion

13: opening portion

14: shaft

15: bearing

16: selection lever

17: camera

18: cable

19: mark

21: hinge mechanism

22: torsion spring

23: tension cable

101{tilde under ( )}103: surgery assistance systems

1. A trocar, comprising:
a pipe portion that inserts a surgical instrument into an interior of a body of a patient;
a head portion that is connected to an upper portion of the pipe portion;
an opening portion that is at a position of the pipe portion that, during surgery, is within the body of the patient;
a camera that is disposed so that it can be changed between a stored position in which it is stored within the pipe portion and a deployed position in which it is deployed via the opening portion to an exterior of the pipe portion and is capable of photography; and
a position mark on the head portion.

2. The trocar according to claim 1, wherein the position mark is an optical mark.

3. A surgery assistance system, comprising:
the trocar according to claim 1, which is a forceps trocar;
a laparoscope having a camera and a position mark;
a position detection sensor that detects positions of a position mark of the laparoscope and a position mark of the forceps trocar; and
an image processing device that estimates the positions of the cameras on a basis of the positions of the position marks, and combines images obtained from the cameras on a basis of the positions of the cameras to create a three dimensional image.

4. A surgery assistance system, comprising:
a plurality of trocars according to claim 1, wherein the trocars are forceps trocars;
a position detection sensor that detects the positions of the position marks of the forceps trocars; and
an image processing device that estimates the positions of the cameras on a basis of the positions of the position marks, and combines images obtained from the cameras on a basis of the positions of the cameras to create a three dimensional image.

5. The surgery assistance system according to claim 3, further comprising a three dimensional projector that is above an operating table, and projects the three dimensional image upon an abdomen of the patient.

6. A port, comprising:
a pipe portion that inserts a surgical instrument into an interior of a chest of a patient;
a head portion that is connected to an upper portion of the pipe portion;
an opening portion that is at a position of the pipe portion that, during surgery, is within a body of the patient;
a camera that is disposed so that it can be changed between a stored position in which it is stored within the pipe portion and a deployed position in which it is deployed via the opening portion to an exterior of the pipe portion and is capable of photography; and
a position mark on the head portion.
